(19) United States
(12) Patent Application Publication
(10) Pub. No.: US 2016/0067168 A1
(43) Pub. Date: Mar. 10, 2016

(54) ORAL AND DENTAL HYGIENE AND CLEANING AGENTS FOR SENSITIVE TEETH

(71) Applicant: Henkel AG & Co. KGaA, Duesseldorf (DE)

(72) Inventors: Thomas Welss, Duesseldorf (DE); Thomas Foerster, Duesseldorf (DE); Markus Semrau, Langenfeld (DE); Kristin Miehlich, Wuppertal (DE); Claudia Hundeiker, Meerbusch (DE)

(21) Appl. No.: 14/944,817

(22) Filed: Nov. 18, 2015

Related U.S. Application Data
(63) Continuation of application No. PCT/EP2014/052038, filed on Feb. 3, 2014.

(30) Foreign Application Priority Data
May 28, 2013 (DE) 10 2013 209 898.7

(51) Int. Cl. A61K 8/85; A61K 8/73; A61K 8/55; A61Q 11/00; A61K 8/25

(52) U.S. Cl. CPC A61K 8/85; A61Q 11/00; A61K 8/25; A61K 8/55; A61K 8/73; A61K 2800/413; A61K 2800/412; A61K 2800/49; A61K 2800/28; A61K 2800/596; A61K 2800/92

(57) ABSTRACT

The invention relates to oral and dental hygiene and cleaning agents which contain, in relation to their weight, between 0.001 and 25% by weight polylactic acid particles and between 1 and 30% by weight precipitated silicic acid(s) with a specific surface area according to ISO 5794-1, Appendix D, of ≦60 m²/g. said agents exhibit a reduced damaging action in comparison to pure silicate-based compositions. In particular, the sensitivity of the teeth during and after the cleaning process is reduced.

### FIELD OF THE INVENTION

The present invention generally relates to oral and dental hygiene and cleaning agents including an active ingredient combination for gentle and effective cleaning of teeth.

### BACKGROUND OF THE INVENTION

Dental cleaning agents are on the market in a variety of forms and serve primarily to clean the dental surface and to prevent diseases of the teeth and gingiva. They usually contain a combination of polishing agents, humectants, surfactants, binders, flavorings and fluoride-containing and antimicrobial active ingredients. In addition to tooth powders which play a lesser role because of their high abrasiveness, dental cleaning agents are available primarily in the form of pastes, creams and translucent or transparent gels. In recent years liquid toothpastes and mouthwashes have become increasingly important.

Many people want to have white teeth and perceive dark or discolored teeth as being cosmetically unacceptable. However, despite regular dental hygiene, it is not always possible to maintain the natural color of teeth. Smoking and dietary habits may cause discoloration of teeth. Bacterial contamination (plaque) on the tooth surface also causes discoloration.

A number of technical solutions for removing or whitening teeth have been developed. Peroxide is the main ingredient used for whitening/bleaching. Peroxide is used in high concentrations in professional bleaching products, but its use in cosmetic products for oral and dental hygiene is limited to 0.1% peroxide. In this concentration, peroxide has only a limited whitening effect and often does not eliminate discoloration of teeth to the desired extent.

Another possibility for whitening teeth consists of effectively removing the dental plaque that causes teeth to appear darker. This method of whitening teeth is also described as “natural whitening.” A high cleaning efficiency is best achieved by using cleaning substances, for example, silica, alumina or calcium carbonate in combination with a surfactant. Unfortunately, toothpastes with an effective system of one or more cleaning substances often have a high abrasiveness, so they result in a certain abrasion of the tooth surface, although it is very minor. This can have a negative effect in particular when the enamel of a tooth is thin anyway, as is the case in people with sensitive teeth. People with sensitive teeth often have teeth with exposed necks, i.e., sections of the tooth in immediate proximity to the gingiva, where no enamel is present as a protective layer and the dentin below that is exposed.

There is therefore a demand for toothpastes that have effective cleaning and whitening effects but at the same time protect and/or remineralize the tooth surface. Such a toothpaste is especially recommended for people with sensitive teeth who do not want to forgo the whitening effect.

The use of polylactic acid particles in products for oral hygiene is described in the international Patent Applications WO2012/177616 and WO2012/177617, where the particles are disclosed exclusively as biodegradable abrasive substances, however. These patent applications do not contain any information about their abrasiveness, and the silicates used in the exemplary compositions are not described in greater detail.

The object of the present invention is to provide preparations for oral and dental hygiene and cleaning, which will result in effective cleaning and whitening, while being less abrasive than traditional toothpastes despite the good cleaning performance.

It has been found that through the combination of polylactic acid particles with certain silicates, the damaging effect can be reduced in comparison with traditional silicate-based references. The sensitivity of the teeth is reduced during and after the cleaning process in particular in comparison with a reference toothpaste.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Oral and dental hygiene and cleaning agent containing—based on its weight—0.001 to 25% by weight polylactic acid particles, and 1 to 30% by weight precipitated silicic acid(s) with a specific surface area according to ISO 5794-1, Appendix D of ≦60 m²/g.

Use of polylactic acid particles to increase the cleaning performance and to improve the protection of the tooth surface from silicate polishing agents.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The subject matter of the invention is oral and dental hygiene and cleaning agents including—based on their weight:
a) 0.001 to 25% by weight polylactic acid particles, and
b) 1 to 30% by weight precipitated silicic acid(s) with a specific surface area according to ISO 5794-1, Appendix D, of ≦60 m²/g.

Oral and dental hygiene agents as well as oral and dental cleaning agents in the sense of the invention include oral and dental powders, oral and dental pastes, liquid oral and dental creams, oral and dental rinses as well as oral and dental gels. Toothpastes and liquid dental cleaning agents are especially suitable here. The oral and dental hygiene and cleaning agents may be in the form of toothpastes, liquid toothpastes, dental powders, mouthwashes or optionally also as a chewable composition, for example, as chewing gum. However, they are preferably present in the form of more or less flowable or plastic toothpastes, such as those used for cleaning teeth with a toothbrush. Another preferred embodiment of the present invention is for mouthwash and mouth rinse solutions that can be used for rinsing out the oral cavity.

The first important ingredient contained in the compositions according to the invention based on weight is 0.001 to 25% by weight polylactic acid particles.

Preferred agents according to the invention use polylactic acid particles within narrower quantity ranges. Oral and dental hygiene and cleaning agents according to the invention here preferably contain—based on their weight—0.002 to 20% by weight, preferably 0.003 to 17.5% by weight, especially preferably 0.004 to 15% by weight, extremely preferably 0.005 to 12.5% by weight and in particular 0.01 to 10% by weight polylactic acid particles.

Polylactic acid, also known as polylactide or PLA, is a name for biodegradable polymers (polyesters) that are accessible primarily by ionic polymerization of lactide, a ring closure of two lactic acid molecules.

A ring-opening polymerization takes place at temperatures between 140 and 180° C. as well as under the influence of catalytic tin compounds (e.g., tin oxide). Therefore, plastics having a high molecular weight and strength are produced. Lactide itself can be produced by fermentation of molasses or by fermentation of glucose with the help of various bacteria.

In addition, high-molecular and pure polylactides can be produced directly from lactic acid with the help of so-called polycondensation. However, disposal of the solvent is problematical in industrial production.

Lactic acid (2-hydroxypropanoic acid) has an asymmetrical carbon atom, so that the polylactic acid also has optically active centers in L(+) and D(−) configurations. The ratio of L monomer units to D monomer units determines the degree of crystallinity, the melting point and the biodegradability of the polymers.

Polylactic acids that are suitable according to the invention include L-polylactic acid, D-polylactic acid and L/D-polylactic acid as well as mixtures thereof. L-Polylactic acid is especially preferred because of its very good biodegradability. In a preferred embodiment of the present invention, the amount by weight of L-lactic acid monomer units in the polylactic acid is greater than 50% by weight, preferably greater than 80% by weight and in particular greater than 90% by weight.

The molecular weight of the polylactic acid is usually 1000 to 1,000,000, preferably 10,000 to 300,000, more preferably 50,000 to 250,000 and in particular from 100,000 to 180,000 Dalton.

In another preferred embodiment of the present invention, the polylactic acid is used in a form that has been blended with fillers. The use of larger amounts of fillers helps to reduce the polymer to particles and increases the biodegradability as well as the internal specific surface area via the porosity and capillarity. Water-soluble fillers are preferred here in particular, for example, metal chlorides such as NaCl, KCl, etc., metal carbonates such as Na₂CO₃, NaHCO₃, etc., metal sulfates such as MgSO₄.

The fillers used may also be natural raw materials, for example, nut shells, wood or bamboo fibers, starch, xanthan, alginates, dextran, agar, etc. These fillers are biodegradable and do not have negative effects on the good ecological properties of the polylactic acid particles.

The biodegradable filler content of the polylactic acid particles may usually be 10 to 70% by weight, with amounts of 20 to 60% by weight being preferred, and 30 to 50% by weight being preferred in particular.

Even if the polylactic acid particles are not used because of their abrasive properties within the scope of the present invention, irregular shapes have proven to be especially preferred because the effect according to the invention can be increased further in comparison with spherical particles.

Especially suitable particles according to the invention have a circularity between 0.1 and 0.6.

The shape of the polylactic acid particles used according to the invention can be defined in various ways, but within the scope of this preferred embodiment of the present invention, the geometric proportions of a particle and—pragmatically—a particle totality can be determined.

Recent high-precision methods allow accurate determination of particle shapes from a large number of particles, usually of more than 10,000 particles, preferably more than 100,000 particles. These methods permit an accurate selection of the average particle shape of a particle totality. The particle shapes are preferably determined by using an Occhio Nano 500 Particle Characterization Instrument with the Callistro version 25 software (Occhio s.a., Li\grave{g}e, Belgium). This device permits preparation, dispersion, imaging and analysis of a particle totality with the device parameters preferably being set as follows: white requested=180, vacuum time=5000 ms, sedimentation time=5000 ms, automatic threshold, number of particles counted/analysis=8000 to 500,000, minimum number of replicates/sample=3, lens setting 1×/1.5×.

The polylactic acid particles that can be used according to the invention preferably have sizes characterized by their equivalent area diameter (ISO 9276-6:2008 (E) section 7) also known as “equivalent circle diameter, ECD” (ASTM F1877-05 section 11.3.2). The average ECD of a particle totality is calculated as the average ECD of each individual particle of a particle totality of at least 10,000 particles, preferably more than 50,000, in particular more than 100,000 particles, after excluding particles with an equivalent circle diameter (ECD) of less than 10 μm from the measurement.

In a preferred embodiment of the present invention, the polylactic acid particles have average ECD values of 10 to 100,000 μm and/or of 50 to 500 μm, more preferably of 100 to 350 μm and in particular of 150 to 250 μm.

Regardless of the average particle size, oral and dental hygiene and cleaning agents according to the invention in which the polylactic acid particles have absolute particle sizes of 1 to 1000 μm, preferably of 2 to 750 μm and in particular of 10 to 500 μm are preferred.

Within the scope of the present invention, shape descriptors, which are calculations from geometric descriptors and/or form factors, are used. Form factors are ratios between two different geometric properties, which are in turn based on a measurement of the proportions of the image of an entire particle or the measurement of the proportions of an ideal geometric body enclosing the particles.

These results are descriptors which are similar to size ratios (aspect ratios).

In a preferred embodiment of the present invention, meso-form descriptors are used to characterize particles. These meso-form descriptors indicate the extent to which a particle deviates from an ideal geometric shape, in particular from a sphere. In the preferred embodiment of the present invention, the polylactic acid particles are different from the typical spherical shape or sphere-like shapes such as granular particles, for example.

The particles here preferably have sharp particles and corners and preferably have concave indentations. Sharp edges of nonspherical particles may be defined as having a radius of less than 20 μm, preferably less than 8 μm and in particular less than 5 μm, wherein the radius is defined as the radius of an imaginary circle following the contour of the corner.

Circularity is a quantitative two-dimensional image analysis and can be determined according to ISO 9276-6:2008 (E), section 8.2. Circularity is a preferred meso-form descriptor and can be determined using the “Occhio Nano 500 Particle Characterization Instrument” described above, for example, with the “Callistro version 25” software (Occhio s.a., Liege, Belgium) or the “Malvern Morphologi G3”. Circularity is occasionally described in the literature as the difference between a particle and a perfect spherical shape. The values for circularity vary between 0 and 1, where 1 describes a perfect sphere or (in a two-dimensional image) a perfect circle:

C=[(4πA)/P²]^{1/2}

where A denotes the projected area (the two-dimensional descriptor) and P denotes the length of the perimeter of the particle.

Polylactic acid particles with an average circularity C of 0.1 to 0.6, preferably of 0.15 to 0.4 and in particular of 0.2 to 0.35 have proven to be particularly suitable within the scope of the present invention. The average values here are obtained by forming a quotient of volume-based measurements and number-based measurements.

Solidity is a quantitative two-dimensional image analysis and can be determined according to ISO 9276-6:2008(E) section 8.2. Solidity is also a preferred meso-form descriptor and can be determined using the Occhio Nano 500 particle characterization instrument described above with the Callistro version 25 software (Occhio s.a., Liege, Belgium) or the Malvern Morphologi G3. Solidity is a meso-form descriptor which describes the concavity of a particle and/or of a particle totality. The values for solidity vary between 0 and 1, where 1 describes a non-concave particle:

Solidity=A/Ac

where A describes the (image) area of the particle and Ac describes the area of the convex shell enclosing the particle.

Polylactic acid particles with an average solidity of 0.4 to 0.9, preferably of 0.5 to 0.8 and in particular of 0.55 to 0.65 have proven to be particularly suitable within the scope of the present invention. The average values here are obtained by forming a quotient of volume-based measurements and number-based measurements.

In particularly preferred embodiments of the present invention, the polylactic acid particles used have an average circularity C of 0.1 to 0.6, preferably of 0.15 to 0.4 and in particular of 0.2 to 0.35 and an average solidity of 0.4 to 0.9, preferably of 0.5 to 0.8 and in particular of 0.55 to 0.65.

The “average” circularities and solidities are average values from measurement of a large number of particles, usually more than 10,000 particles, preferably more than 50,000 particles and in particular more than 100,000 particles, where particles with an equivalent circle diameter (ECD) of less than 10 μm are excluded from the measurement.

After being produced, the polylactic acid polymer can be converted to the desired particle size and shape by milling methods, for example.

A particularly preferred method for producing the particles of the desired circularity and solidity is to produce a foam of polylactic acid and then to mill it.

Even if the polylactic acid particles within the scope of the present invention are not used because of their abrasive properties, polylactic acid particles of a certain hardness have proven to be particularly preferred because the effect according to the invention can be further enhanced with these particles in comparison with particles that are too hard or too soft.

The hardness of the particles can be varied by the ratio of D to L monomers and by the molecular weight.

Preferred polylactic acid particles have a hardness of 3 to 50 kg/mm², preferably of 4 to 25 kg/mm² and in particular of 5 to 15 kg/mm² on the HV Vickers hardness scale.

As the second essential ingredient, the compositions according to the invention contain, based on their weight, 1 to 30% by weight precipitated silicic acid(s) with a specific surface area according to ISO 5794-1, Appendix D of ≦60 m²/g. The precipitated silicic acids, which have corresponding specific surface areas, are preferably used within narrower quantity ranges and in particular preferably precipitated silicic acids having even lower specific surface areas according to ISO 5794-1, Appendix D are used. Preferred oral and dental hygiene and cleaning agents according to the invention contain 2.5 to 25% by weight, preferably 5 to 20% by weight, especially preferably 7.5 to 17.5% by weight, more preferably 8.0 to 15.0% by weight and in particular 10.0 to 14.0% by weight precipitated silicic acid(s) with a specific surface area according to ISO 5794-1, Appendix D of ≦55 m²/g.

Especially preferred oral and dental hygiene and cleaning agents according to the invention are characterized in that all the precipitated silicic acid(s) contained in the agent have a specific surface area of ≦60 m²/g according to ISO 5794-1, Appendix D, preferably ≦52.5 m²/g, more preferably ≦49 m²/g and in particular ≦47 m²/g.

In further preferred agents according to the invention, the precipitated silicic acids used are characterized by additional physical parameters. Precipitated silicic acids to be used preferably have a tamped density of >360 g/L (measured according to ISO 787-11), especially preferably >375 g/L, more preferably >400 g/L and in particular >425 g/L.

It is additionally preferable to use precipitated silicic acids having a DBP absorption of less than 140 g/100 g according to DIN 53601. Most especially preferred precipitated silicic acids to be used according to the invention have a DBP absorption of less than 135 g/100 g according to DIN 53601, preferably a DBP absorption of less than 130 g/100 g according to DIN 53601 and in particular of less than 125 g/100 g.

Especially preferred agents according to the invention contain 2.5 to 25% by weight, preferably 5 to 20% by weight, especially preferably 7.5 to 17.5% by weight, more preferably 8.0 to 15.0% by weight and in particular 10.0 to 14.0% by weight precipitated silicic acid(s) with a specific surface area of ≦45 m²/g according to ISO 5794-1, Appendix D and a tamped density (measured according to ISO 787-11) of >425 g/L.

Further preferred agents according to the invention contain 2.5 to 25% by weight, preferably 5 to 20% by weight, especially preferably 7.5 to 17.5% by weight, more preferably 8.0 to 15.0% by weight and in particular 10.0 to 14.0% by weight precipitated silicic acid(s) with a specific surface area of ≦45 m²/g according to ISO 5794-1, Appendix D and a DBP absorption of less than 125 g/100 g according to DIN 53601.

In particular, preferred agents according to the invention contain 2.5 to 25% by weight, preferably 5 to 20% by weight, especially preferably 7.5 to 17.5% by weight, more preferably 8.0 to 15.0% by weight and in particular 10.0 to 14.0% by weight precipitated silicic acid(s) with a specific surface area of ≦45 m²/g according to ISO 5794-1, Appendix D and a tamped density of >425 g/L (measured according to ISO 787-11) and a DBP absorption of less than 125 g/100 g according to DIN 53601.

The agents according to the invention may also contain polishing agents in addition to the aforementioned precipitated silicic acids a). Suitable polishing agents include in principle all friction bodies known for toothpaste, in particular those that do not contain any calcium ions. Preferred suitable polishing agent components therefore include aluminum hydroxide, aluminum oxide, sodium aluminosilicates, organic polymers or mixtures of such friction bodies.

It is preferable for the compositions according to the invention to contain little to no precipitated silicic acids having a specific surface area of >55 m²/g according to ISO 5794-1, Appendix D. If such silicic acids are to be used, the weight ratio of precipitated silicic acids with a specific surface area of ≦55 m²/g (ingredient a)) according to ISO 5794-1, Appendix D to precipitated silicic acids with a specific surface area according to ISO 5794-1, Appendix D of >55 m²/g, preferably >1:1, more preferably >2:1, even more preferably >5:1, especially preferably >10:1 and in particular >50:1.

As an additional polishing agent component, for example, aluminum oxide may be present in the form of weakly calcined alumina including—and—aluminum oxide in an amount of approximately 1-5% by weight. One suitable aluminum oxide of this type is available under the brand name Poliertonerde P10 feinst [ultrafine polishing alumina P10] (Giulini Chemie). In addition, all abrasive substances known for toothpaste are also suitable as polishing agents, for example, sodium aluminum silicates, e.g., zeolite A, organic polymers such as polymethacrylate or mixtures of these and the abrasive substances mentioned above.

As an additional ingredient, the agents according to the invention may contain 0.001 to 10.0% by weight of at least one calcium salt. Calcium salt(s) are especially preferably used within narrower quantity ranges, so that preferred oral and dental hygiene and cleaning agents contain 0.05 to 7.5% by weight, preferably 0.1 to 5% by weight, more preferably 0.15 to 2.5% by weight and in particular 0.2 to 1.25% by weight calcium salt(s).

According to the invention, all physiologically tolerable calcium salts can be used, but it is preferable to use calcium salts that manifest an additional benefit in the oral and dental hygiene and cleaning agents according to the invention. Of these compounds, calcium hydrogen phosphate dihydrate and/or calcium glycerophosphate are most particularly preferred.

According to the literature, calcium hydrogen phosphate dihydrate CaHPO₄.2H₂O is also referred to as brushite or as dicalcium phosphate dihydrate. Use of CaHPO₄.2H₂O, which is described by CAS No. 7789-77-7, is especially preferred according to the invention.

Oral and dental hygiene and cleaning agents according to the invention including calcium hydrogen phosphate dihydrate CaHPO₄.2H₂O have definite advantages in abrasiveness in comparison with other agents, so that gentle cleaning of sensitive teeth is even better when using the combination of polylactic acid, a defined silicate and calcium hydrogen phosphate dihydrate as the calcium salt according to the invention.

It has been found that calcium hydrogen phosphate dihydrate CaHPO₄.2H₂O is preferably used within narrow quantity ranges. Preferred oral and dental hygiene and cleaning agents according to the invention contain 0.25 to 7.5% by weight, preferably 0.5 to 7.0% by weight, especially preferably 1.0 to 6.0% by weight, more preferably 2.0 to 5.0% by weight and in particular 3.5 to 4.5% by weight calcium hydrogen phosphate dihydrate.

In addition to or instead of calcium hydrogen phosphate dihydrate, the agents according to the invention may especially preferably contain calcium glycerophosphate, i.e., a calcium salt of at least one glycerophosphoric acid.

Glycerophosphoric acid is a dibasic acid that occurs in two isomeric forms, depending on whether the phosphoric acid group is bound to a terminal OH group of the glycerol or to a medial OH group. The form in which the phosphoric acid group is bound to a terminal OH group of glycerol is also known as the alpha isomer. The form in which the phosphoric acid group is bound to the medial OH group of glycerol is also known as the beta isomer.

The alpha isomer is also optically active and occurs in the two enantiomeric forms of sn-glycerol-1-phosphoric acid

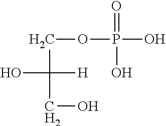

as well as sn-glycerol-3-phosphoric acid.

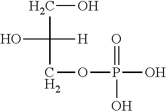

The prefix sn in glycerol derivatives stands for “stereospecifically numbered” and requires the 2-hydroxy group to face to the left in the Fischer projection used above. Glycerol-2-phosphate is not optically active. The glycerophosphoric acids are approximately as strong as phosphoric acid.

Use of the alpha isomer is preferred according to the invention, regardless of which of the two enantiomers is used. If it is desirable to use enantiomer-pure compounds, then the calcium salt of sn-glycerol-3-phosphoric acid is preferably used.

In summary, oral and dental hygiene and cleaning agents according to the invention including calcium salts of glycerophosphoric acids of formulas (Ia) and (Ib) are preferred:

HO—CH₂—CH(OH)—CH₂—OP(O)O₂²⁻Ca²⁺ (Ia)

HO—CH₂—CH(OP(O)O₂²⁻)—CH₂—OHCa²⁺ (Ib).

Especially preferred oral and dental hygiene and cleaning agents according to the invention are characterized in that the weight ratio of the calcium salts of formulas (Ia) to (Ib) is greater than 50:50, preferably greater than 60:40, especially preferably greater than 70:30 and in particular greater than 80:20.

Use of calcium glycerophosphates within narrower quantity ranges is preferred. Preferred oral and dental hygiene and cleaning agents according to the invention contain—based on their weight—0.01 to 2.5% by weight, preferably 0.05 to 2.0% by weight, especially preferably 0.1 to 1.0% by weight, more preferably 0.11 to 0.75% by weight and in particular 0.12 to 0.5% by weight calcium glycerophosphate.

Most especially preferred agents according to the invention contain 0.25 to 7.5% by weight, preferably 0.5 to 7.0% by weight, especially preferably 1.0 to 6.0% by weight, more preferably 2.0 to 5.0% by weight and in particular 3.5 to 4.5% by weight calcium hydrogen phosphate dihydrate and 0.01 to 2.5% by weight, preferably 0.05 to 2.0% by weight, especially preferably 0.1 to 1.0% by weight, more preferably 0.11 to 0.75% by weight and in particular 0.12 to 0.5% by weight calcium glycerophosphate.

Oral and dental hygiene and cleaning agents according to the invention may contain additional ingredients. It has been found that certain magnesium salts can further increase the synergistic effect of the combination according to the invention. Accordingly, especially preferred oral and dental hygiene and cleaning agents according to the invention are those including magnesium sulfate heptahydrate in the amount of 0.25 to 7.5% by weight, preferably 0.5 to 6.0% by weight, especially preferably 1.0 to 5.0% by weight, more preferably 1.5 to 3.0% by weight and in particular 1.75 to 2.5% by weight. When calcium hydrogen phosphate dihydrate is used as the calcium salt, it is especially preferable to use magnesium sulfate heptahydrate.

In especially preferred oral and dental hygiene and cleaning agents, the weight ratio of the magnesium sulfate heptahydrate to calcium hydrogen phosphate dihydrate is 20:1 to 1:1, preferably 15:1 to 2:1, more preferably 10:1 to 5:2, especially preferably 4:1 to 3:1 and in particular 18:7 to 15:7.

Surface-active substances can also be used in the agents according to the invention. They serve to support the cleaning effect in toothpastes, for example, and, if desired, also serve to develop foam for toothbrushes or for mouthwashes as well as to stabilize the dispersion of polishing substance in the carrier and are generally used in the amount of 0.1-5% by weight in mouthwash solutions as well as in toothpastes.

Suitable surfactants include, for example, linear sodium alkyl sulfates with 12-18 carbon atoms in the alkyl group. These substances additionally have an enzyme-inhibiting effect on the bacterial metabolism of dental plaque. Other suitable surfactants include alkali salts, preferably sodium salts of alkyl polyglycol ether sulfate with 12-16 carbon atoms in the linear alkyl group and 2-6 glycol ether groups in the molecule, of linear alkane (C₁₂-C₁₈) sulfonate, of sulfosuccinic acid monoalkyl (C₁₂-C₁₈) esters, of sulfated fatty acid monoglycerides, sulfated fatty acid alkanolamides, sulfoacetic acid alkyl (C₁₂-C₁₆) esters, acyl sarcosines, acyl taurides and acyl isothionates, each with 8-18 carbon atoms in the acyl group. Zwitterionic, ampholytic and nonionic surfactants are also suitable, e.g., ethoxylates of fatty acid mono- and diglycerides, of fatty acid sorbitan esters and alkyl (oligo)glucosides, as well as fatty acid amidoalkylbetaines.

According to the invention, it is preferable to largely restrict the use of surfactants in order to allow the desensitizing effect of the combination according to the invention to be manifested even more definitely. Therefore, oral and dental hygiene and cleaning agents according to the invention that contain—based on their weight—less than 5% by weight, preferably less than 4% by weight, especially preferably less than 3% by weight and in particular less than 2% by weight surfactant(s) are especially preferred according to the invention.

It is most especially preferred to largely limit the use of anionic surfactants in particular or to omit these surfactants entirely. Preferred oral and dental hygiene and cleaning agents according to the invention here are characterized in that they contain less than 2% by weight, preferably less than 1% by weight, especially preferably less than 0.5% by weight and in particular less than 0.1% by weight anionic surfactant(s), wherein preferred agents are free of anionic surfactants.

If surfactants are to be used—preferably within the maximum limits stipulated above—the use of amphoteric surfactants is preferred. Preferred oral and dental hygiene and cleaning agents according to the invention that contain surfactant(s) contain 0.1 to 5% by weight, preferably 0.25 to 4% by weight, especially preferably 0.5 to 3.0% by weight, more preferably 0.75 to 2.0% by weight and in particular 1.0 to 1.5% by weight amphoteric surfactant(s).

Particularly preferred oral and dental hygiene and cleaning agents according to the invention of this embodiment includes 0.1 to 5% by weight, preferably 0.2 to 4% by weight, especially preferably 0.25 to 3% by weight, more preferably 0.3 to 2% by weight and in particular 0.4 to 0.8% by weight cocoamidopropylbetaine.

The oral and dental hygiene and cleaning agents according to the invention may also contain other ingredients. Use of so-called humectants which prevent drying in the case of toothpastes is preferred here. With so-called liquid toothpastes having a flowable rheology, these ingredients serve as a matrix and are used in larger amounts. In the case of mouthwash and mouth rinse, these alcohols serve as consistency regulators and additional sweeteners.

Oral and dental hygiene and cleaning agents according to the invention here that contain—based on their weight—0.5 to 60% by weight, preferably 0.75 to 55% by weight, especially preferably 1 to 50% by weight and in particular 2 to 40% by weight of at least one polyvalent alcohol from the group of sorbitol and/or glycerol and/or 1,2-propylene glycol % or mixtures thereof are preferred.

For certain application ranges, it may be advantageous to use just one of the three ingredients listed above. In most cases sorbitol is preferred, but mixtures of two of the three substances or all three substances may be preferred in other application fields. A mixture of glycerol, sorbitol and 1,2-propylene glycol in a weight ratio of 1:(0.5-1):(0.1-0.5) has proven to be particularly advantageous here.

In addition to sorbitol and/or glycerol and/or 1,2-propylene glycol, additional polyvalent alcohols that are suitable here include those having at least two OH groups, preferably mannitol, xylitol, polyethylene glycol, polypropylene glycol and mixtures thereof. Of these compounds, those with two to 12 OH groups and in particular those with 2, 3, 4, 5, 6 or 10 OH groups are preferred.

Polyhydroxy compounds with two OH groups include, for example, glycol (CH₂(OH)CH₂OH) and other 1,2-diols such as H—(CH₂)ₙ—CH(OH)CH₂OH where n=2, 3, 4, 5, 6, 7, 8, 9, 10, 11, 12, 13, 14, 15, 16, 17, 18, 19, 20. 1,3-Diols such as H—(CH₂)ₙ—CH(OH)CH₂CH₂OH, where n=1, 2, 3, 4, 5, 6, 7, 8, 9, 10, 11, 12, 13, 14, 15, 16, 17, 18, 19, 20 can also be used according to the invention. The (n, n+1)- and/or (n, n+2)-diols with nonterminal OH groups may also be used. Important representatives of the polyhydroxy compounds having two OH groups also include the polyethylene and polypropylene glycols. Additional polyvalent alcohols that may be use include, for example, xylitol, propylene glycols, polyethylene glycols, in particular those with an average molecular weight of 200-800.

Use of sorbitol is especially preferred, so that agents, which do not contain any other polyvalent alcohols in addition to sorbitol, are especially preferred.

The agents according to the invention may additionally also contain wound-healing and anti-inflammatory substances, for example, active ingredients to prevent gingivitis. Such substances can be selected, for example, from allantoin, azulene, chamomile extracts, tocopherol, panthenol, bisabolol, sage extracts.

Oral and dental hygiene and cleaning agents may also contain other substances that are effective against plaque and/or dental calculus.

Substances that are effective against dental calculus may include, for example, chelating agents such as ethylenediaminetetraacetic acid and its sodium salts, phosphate salts such as the water-soluble dialkali or tetraalkali metal pyrophosphate salts, e.g., Na₄P₂O₇, K₄P₂O₇, Na₂K₂P₂O₇, Na₂H₂P₂O₇ and K₂H₂P₂O₇ or polyphosphate salts which may be selected, for example, from water-soluble alkali metal tripolyphosphates such as sodium tripolyphosphate and potassium tripolyphosphate.

Oral and dental hygiene and cleaning agents that are preferred according to the invention are characterized in that they additionally contain phosphate(s), preferably alkali metal phosphate(s) and in particular sodium tripolyphosphate, preferably in amounts of 1 to 10% by weight, especially preferably of 2 to 8% by weight and in particular of 3 to 7% by weight, each based on the total agent.

For example, natural and/or synthetic water-soluble polymers such as alginates, carraghenates, gum tragacanth, starch and starch ethers, cellulose ethers such as, for example, carboxymethyl cellulose (sodium salt), hydroxyethyl cellulose, methyl hydroxypropyl cellulose, guar gum, acacia gum, agar, xanthan gum, succinoglycan gum, carob bean powder, pectins, water-soluble carboxyvinyl polymers (e.g., Carbopol® products), polyvinyl alcohol, polyvinylpyrrolidone, polyethylene glycols, in particular those with molecular weights of 1500-1,000,000.

Additional substances that are suitable for controlling viscosity include, for example, sheet silicates such as montmorillonite clays, colloidal thickening silicic acids such as aerogel silicic acids, pyrogenic silicic acids or extremely finely milled precipitated silicic acids. Viscosity stabilizing additives from the group of cationic, zwitterionic or ampholytic nitrogen-containing surfactants, the hydroxypropyl-substituted hydrocolloids or polyethylene glycol/polypropylene glycol copolymers having an average molecular weight of 1000 to 5000 or a combination of the aforementioned compounds may also be used in the toothpastes.

Xanthan is especially compatible with the combination according to the invention. Agents according to the invention including xanthan are extremely stable in storage and have pleasant product haptics.

Preferred oral and dental hygiene and cleaning agents according to the invention are therefore characterized in that they additionally contain 0.1 to 7.5% by weight, preferably 0.25 to 5% by weight, more preferably 0.5 to 2.5% by weight and in particular 0.6 to 1.5% by weight xanthan.

In addition to the aforementioned obligatory components, the dental hygiene agents according to the invention may also contain other essentially known excipients and additives. On the whole, the dental cleaning agents according to the invention may contain the usual excipients and additives in amounts up to 10% by weight.

The organoleptic properties of the dental hygiene agents according to the invention can be improved by adding aromatic oils and sweeteners.

All the usual natural and synthetic flavorings used in oral and dental hygiene agents may be used as the aromatic oils here. Natural flavorings may be contained in the form of the natural essential oils isolated from herbs as well as the individual components isolated therefrom.

Suitable flavorings include, for example, peppermint oil, spearmint oil, eucalyptus oil, anise oil, fennel oil, caraway oil, menthyl acetate, cinnamaldehyde, anethol, vanillin, thymol as well as mixtures of these components.

Suitable sweeteners include, for example, sodium saccharin, sodium cyclamate, sucrose, lactose, maltose, and fructose.

Other conventional additives and excipients for toothpaste and mouthwash or mouth rinse solutions include:
surface-active substances, preferably anionic, zwitterionic, amphoteric, nonionic surfactants or a combination of several different surfactants,
solvents and solubilizers, for example, low monovalent or polyvalent alcohols or ethers, e.g., ethanol, 1,2-propylene glycol, diethylene glycol or butyl diglycol,
pigments such as titanium dioxide,
dyes,
buffer substances, e.g., primary, secondary or tertiary alkali phosphates or citric acid/sodium citrate,
other wound-healing or anti-inflammatory substances, e.g., allantoin, urea, azulene, chamomile active ingredients, acetylsalicylic acid derivatives or thiocyanates,
additional vitamins such as ascorbic acid, biotin, tocopherol or rutin,
mineral salts such as manganese, zinc or magnesium salts.

It has been found that the efficiency of the oral and dental hygiene and cleaning agents according to the invention can be further increased if the agents contain salivation-promoting substances. In particular the antibacterial effect and, with it, the anti-caries effect and the effect against gingivitis and/or periodontitis are strengthened in this way.

Salivation is understood to be the production and release of saliva, including unphysiologically elevated levels in the broader sense. Substances that stimulate saliva flow and increase the amount and/or release of saliva may originate from a wide variety of different classes of substances.

Pilocarpine is one example of a substance that is suitable according to the invention and may be present in the oral and dental hygiene and cleaning agents according to the invention.

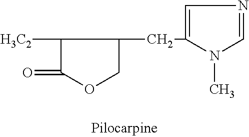

Additional salivation-promoting substances include in particular so-called sharp substances, i.e., sharp tasting substances and/or those that create a feeling of heat. Preferred oral and dental hygiene and cleaning agents according to the invention are characterized in that they contain at least one sharp tasting substance and/or a substance that imparts a feeling of heat as the salivation-promoting substance.

The products of this embodiment according to the invention contain a sharp-tasting substance and/or a substance that imparts a feeling of heat as the salivation-promoting ingredient. These substances impart to the user a sharp, tingling, mouth-watering or heat-producing effect, i.e., they give a sensory impression of heat or burning or prickling, tingling, tickling or frizzing and thereby promote saliva flow.

Products of this embodiment that are preferred according to the invention contain the sharp tasting substances and/or the substances that create a feeling of heat in amounts of 0.00001 to 5% by weight, preferably from 0.0005 to 2.5% by weight, more preferably from 0.001 to 1% by weight, especially preferably from 0.005 to 0.75% by weight and in particular from 0.01 to 0.5% by weight, each based on the weight of the total agent.

As a sharp tasting substance or a substance that creates a feeling of heat, a number of substances may be used. N-Alkyl-substituted amides of unsaturated carboxylic acids, for example, the following are preferred in particular:
2E,4E-decadienoic acid N-methylamide
2E,4E-decadienoic acid N-ethylamide
2E,4E-decadienoic acid N-n-propylamide
2E,4E-decadienoic acid N-isopropylamide
2E,4E-decadienoic acid N-n-butylamide
2E,4E-decadienoic acid N-(1-methylpropyl)amide
2E,4E-decadienoic acid N-isobutylamide
2E,4E-decadienoic acid N-tert-butylamide
2E,4Z-decadienoic acid N-methylamide
2E,4Z-decadienoic acid N-ethylamide
2E,4Z-decadienoic acid N-n-propylamide
2E,4Z-decadienoic acid N-isopropylamide
2E,4Z-decadienoic acid N-n-butylamide
2E,4Z-decadienoic acid N-(1-methylpropyl)amide
2E,4Z-decadienoic acid N-isobutylamide
2E,4Z-decadienoic acid N-tert-butylamide
2E,4E,8Z-decatrienoic acid N-methylamide
2E,4E,8Z-decatrienoic acid N-ethylamide
2E,4E,8Z-decatrienoic acid N-n-propylamide
2E,4E,8Z-decatrienoic acid N-isopropylamide
2E,4E,8Z-decatrienoic acid N-n-butylamide
2E,4E,8Z-decatrienoic acid N-(1-methylpropyl)amide
2E,4E,8Z-decatrienoic acid N-isobutylamide
2E,4E,8Z-decatrienoic acid N-tert-butylamide
2E,4Z,8Z-decatrienoic acid N-methylamide
2E,4Z,8Z-decatrienoic acid N-ethylamide
2E,4Z,8Z-decatrienoic acid N-n-propylamide
2E,4Z,8Z-decatrienoic acid N-isopropylamide
2E,4Z,8Z-decatrienoic acid N-n-butylamide
2E,4Z,8Z-decatrienoic acid N-(1-methylpropyl)amide
2E,4Z,8Z-decatrienoic acid N-isobutylamide
2E,4Z,8Z-decatrienoic acid N-tert-butylamide
2E,4E,8E-decatrienoic acid N-methylamide
2E,4E,8E-decatrienoic acid N-ethylamide
2E,4E,8E-decatrienoic acid N-n-propylamide
2E,4E,8E-decatrienoic acid N-isopropylamide
2E,4E,8E-decatrienoic acid N-n-butylamide
2E,4E,8E-decatrienoic acid N-(1-methylpropyl)amide
2E,4E,8E-decatrienoic acid N-isobutylamide
2E,4E,8E-decatrienoic acid N-tert-butylamide
2E,4Z,8E-decatrienoic acid N-methylamide
2E,4Z,8E-decatrienoic acid N-ethylamide
2E,4Z,8E-decatrienoic acid N-n-propylamide
2E,4Z,8E-decatrienoic acid N-isopropylamide
2E,4Z,8E-decatrienoic acid N-n-butylamide
2E,4Z,8E-decatrienoic acid N-(1-methylpropyl)amide
2E,4Z,8E-decatrienoic acid N-isobutylamide
2E,4Z,8E-decatrienoic acid N-tert-butylamide
2E,6Z,8E-decatrienoic acid N-methylamide
2E,6Z,8E-decatrienoic acid N-ethylamide
2E,6Z,8E-decatrienoic acid N-n-propylamide
2E,6Z,8E-decatrienoic acid N-isopropylamide
2E,6Z,8E-decatrienoic acid N-n-butylamide
2E, 6Z,8E-decatrienoic acid N-(1-methylpropyl)amide
2E,6Z,8E-decatrienoic acid N-isobutylamide
2E,6Z,8E-decatrienoic acid N-tert-butylamide
2E,6E,8E-decatrienoic acid N-methylamide
2E,6E,8E-decatrienoic acid N-ethylamide
2E,6E,8E-decatrienoic acid N-n-propylamide
2E,6E,8E-decatrienoic acid N-isopropylamide
2E,6E,8E-decatrienoic acid N-n-butylamide
2E,6E,8E-decatrienoic acid N-(1-methylpropyl)amide
2E,6E,8E-decatrienoic acid N-isobutylamide
2E,6E,8E-decatrienoic acid N-tert-butylamide
2E,6Z,8Z-decatrienoic acid N-methylamide
2E,6Z,8Z-decatrienoic acid N-ethylamide
2E,6Z,8Z-decatrienoic acid N-n-propylamide
2E,6Z,8Z-decatrienoic acid N-isopropylamide
2E,6Z,8Z-decatrienoic acid N-n-butylamide
2E,6Z,8Z-decatrienoic acid N-(1-methylpropyl)amide
2E,6Z,8Z-decatrienoic acid N-isobutylamide
2E,6Z,8Z-decatrienoic acid N-tert-butylamide
2E,6E,8Z-decatrienoic acid N-methylamide
2E,6E,8Z-decatrienoic acid N-ethylamide
2E,6E,8Z-decatrienoic acid N-n-propylamide
2E,6E,8Z-decatrienoic acid N-isopropylamide
2E,6E,8Z-decatrienoic acid N-n-butylamide
2E,6E,8Z-decatrienoic acid N-(1-methylpropyl)amide
2E,6E,8Z-decatrienoic acid N-isobutylamide
2E,6E,8Z-decatrienoic acid N-tert-butylamide
2E,7Z,9E-undecatrienoic acid N-methylamide
2E,7Z,9E-undecatrienoic acid N-ethylamide
2E,7Z,9E-undecatrienoic acid N-n-propylamide
2E,7Z,9E-undecatrienoic acid N-isopropylamide
02E,7Z,9E-undecatrienoic acid N-n-butylamide
2E,7Z,9E-undecatrienoic acid N-(1-methylpropyl)amide
2E,7Z,9E-undecatrienoic acid N-isobutylamide
2E,7Z,9E-undecatrienoic acid N-tert-butylamide
2E,7E,9E-undecatrienoic acid N-methylamide
2E,7E,9E-undecatrienoic acid N-ethylamide
2E,7E,9E-undecatrienoic acid N-n-propylamide
2E,7E,9E-undecatrienoic acid N-isopropylamide
2E,7E,9E-undecatrienoic acid N-n-butylamide
2E,7E,9E-undecatrienoic acid N-(1-methylpropyl)amide
2E,7E,9E-undecatrienoic acid N-isobutylamide
2E,7E,9E-undecatrienoic acid N-tert-butylamide
2E,7Z,9Z-undecatrienoic acid N-methylamide
2E,7Z,9Z-undecatrienoic acid N-ethylamide
2E,7Z,9Z-undecatrienoic acid N-n-propylamide
2E,7Z,9Z-undecatrienoic acid N-isopropylamide
2E,7Z,9Z-undecatrienoic acid N-n-butylamide
2E,7Z,9Z-undecatrienoic acid N-(1-methylpropyl)amide
2E,7Z,9Z-undecatrienoic acid N-isobutylamide
2E,7Z,9Z-undecatrienoic acid N-tert-butylamide
2E,7Z,9E-undecatrienoic acid N-methylamide
2E,7Z,9E-undecatrienoic acid N-ethylamide
2E,7Z,9E-undecatrienoic acid N-n-propylamide
2E,7Z,9E-undecatrienoic acid N-isopropylamide
2E,7Z,9E-undecatrienoic acid N-n-butylamide
2E,7Z,9E-undecatrienoic acid N-(1-methylpropyl)amide
2E,7Z,9E-undecatrienoic acid N-isobutylamide
2E,7Z,9E-undecatrienoic acid N-tert-butylamide

Other substitution patterns on the nitrogen atom are of course also possible and preferred. For example, longer-chain n-alkyl radicals ( . . . —N-n-pentylamide, . . . —N-n-pentylamide, . . . —N-n-pentylamide, . . . —N-n-pentylamide, . . . —N-n-pentylamide, . . . —N-n-hexylamide, . . . —N-n-heptylamide, . . . —N-n-octylamide, . . . —N-n-nonylamide, . . . —N-n-decylamide, . . . —N-n-undecylamide, . . . —N-n-dodecylamide, . . . —N-n-tridecylamide, etc.) or disubstituted . . . —N,N-dialkylamides such as . . . —N,N-dimethylamide, . . . —N,N-diethylamide, . . . —N,N-di-n-propylamide, . . . —N,N-diisopropylamide, . . . —N,N-di-n-butylamide, . . . —N,N-di(1-methylpropyl)amide, . . . —N,N-diisobutylamide, . . . —N,N-di-tert-butylamide, . . . —N,N-methylethylamide, . . . —N,N-methyl-n-propylamide, . . . —N,N-methylisopropylamide, . . . —N,N-ethyl-n-propylamide, . . . —N,N-ethylisopropylamide, etc.

Of the compounds listed, a few of them are especially preferred within the scope of the present invention. These are listed below:

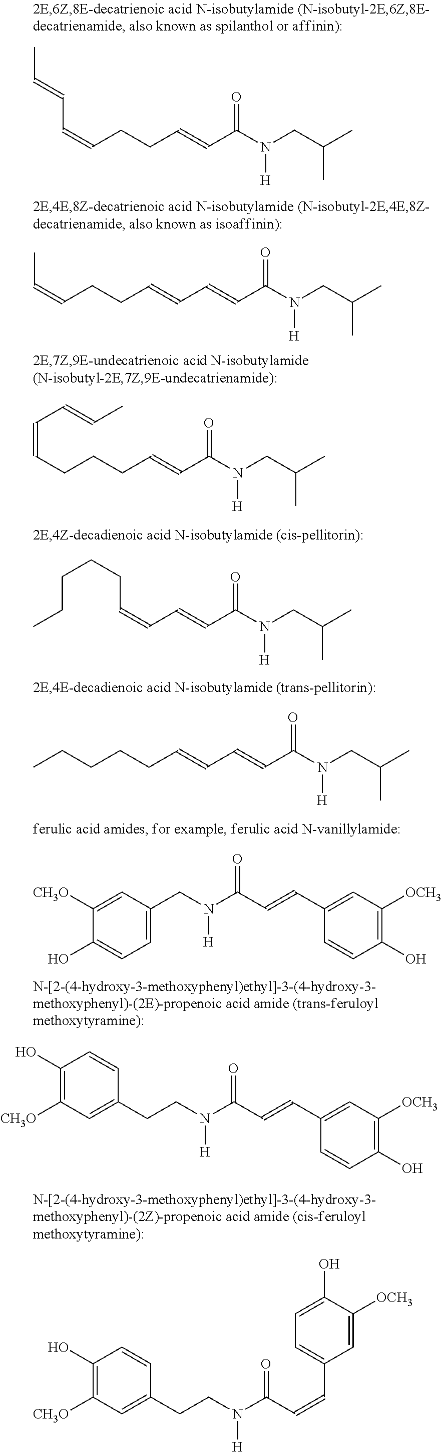

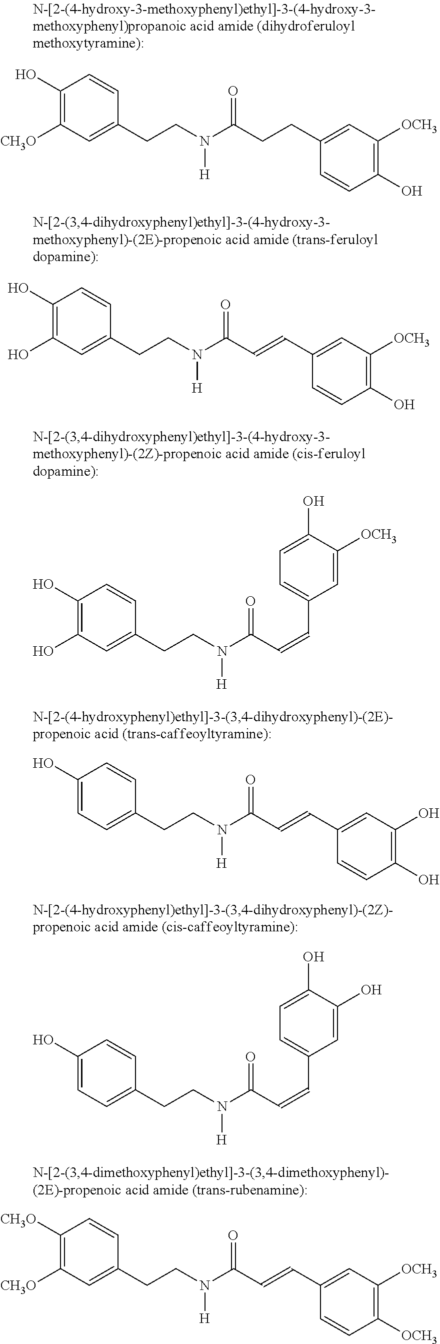

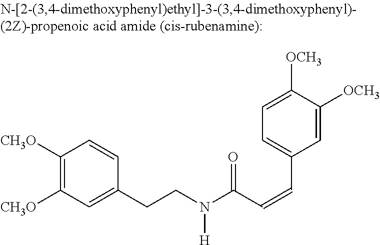

Additional sharp substances that can be used with particular preference within the scope of the present invention include, for example, extracts of natural plants. Sharp-tasting plant extracts may include all physiologically safe plant extracts that cause a sharp or warm sensory impression. Sharp-tasting plant extracts that are preferred include, for example, pepper extract (Piper ssp., in particular Piper nigrum), water pepper extract (Polygonum ssp., in particular Polygonum hydropiper), extracts of Allium ssp. (in particular onion and garlic extracts), radish extracts (Raphanus ssp.), horseradish extracts (Cochlearia armoracia), extracts of black mustard (Brassica nigra), wild or yellow mustard (Sinapis ssp., in particular Sinapis arvensis and Sinapis alba), bertram root extract (Anacyclus ssp., in particular Anacyclus pyrethrum L.), echinacea extracts (Echinaceae ssp.), Szechuan pepper extracts (Zanthoxylum ssp., in particular Zanthoxylum piperitum), spilanthes extract (Spilanthes ssp., in particular Spilanthes acmella), chili pepper extract (Capsicum ssp., in particular Capsicum frutescens), grains of paradise extract (Aframomum ssp., in particular Aframomum melegueta [Rose] K. Schum.), ginger extract (Zingiber ssp., in particular Zingiber officinale) and galanga extract (Kaempferia galanga or Alpinia galanga).

Gingerol obtained from ginger extract is an especially suitable substance. N-Ethyl-p-menthane-3-carboxamide(N-ethyl-5-methyl-2-isopropylcyclohexanecarboxamide) can also be used.

Other substances that have a sharp taste or create a feeling of heat may include, for example, capsaicin, dihydrocapsaicin, gingerol, paradol, shogaol, piperine, carboxylic acid N-vanillylamides, in particular nonanoic acid N-vanillylamide, 2-alkenoic acid amides, in particular 2-nonenoic acid N-isobutylamide, 2-nonenoic acid N-4-hydroxy-3-methoxy-phenylamide, alkyl ethers of 4-hydroxy-3-methoxybenzyl alcohol, in particular 4-hydroxy-3-methoxybenzyl-n-butyl ether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylenedioxybenzyl alcohol, nicotinaldehyde, methyl nicotinate, propyl nicotinate, 2-butoxyethylnicotinate, benzyl nicotinate, 1-acetoxychavicol, polygodial or isodrimeninol.

Preferred remineralizing products according to the invention are characterized in that they contain at least one sharp substance from the group of N-alkyl-substituted amides of unsaturated carboxylic acids, preferably
a. 2E,6Z,8E-decatrienoic acid N-isobutylamide (spilanthol) and/or
b. 2E,4E,8Z-decatrienoic acid N-isobutylamide and/or
c. 2E,7Z,9E-undecatrienoic acid N-isobutylamide and/or
d. 2E,4Z-decadienoic acid N-isobutylamide (cis-pellitorin) and/or
e. 2E,4E-decadienoic acid N-isobutylamide (trans-pellitorin) and/or
f. Ferulic acid N-vanillylamide and/or
g. N-[2-(4-hydroxy-3-methoxyphenyl)ethyl]-3-(4-hydroxy-3-methoxyphenyl)-(2E)-propenoic acid amide (trans-feruloyl methoxytyramine) and/or
h. N-[2-(4-hydroxy-3-methoxyphenyl)ethyl]-3-(4-hydroxy-3-methoxyphenyl)-(2Z)-propenoic acid amide (cis-feruloyl methoxytyramine) and/or
i. N-[2-(4-hydroxy-3-methoxyphenyl)ethyl]-3-(4-hydroxy-3-methoxyphenyl)propanoic acid amide (dihydroferuloyl methoxytyramine) and/or
j. N-[2-(3,4-dihydroxyphenyl)ethyl]-3-(4-hydroxy-3-methoxyphenyl)-(2E)-propenoic acid amide (trans-feruloyl dopamine) and/or
k. N-[2-(3,4-dihydroxyphenyl)ethyl]-3-(4-hydroxy-3-methoxyphenyl)-(2Z)-propenoic acid amide (cis-feruloyl dopamine) and/or
l. N-[2-(4-hydroxyphenyl)ethyl]-3-(3,4-dihydroxyphenyl)-(2E)-propenoic acid (trans-caffeoyltyramine) and/or
m. N-[2-(4-hydroxyphenyl)ethyl]-3-(3,4-dihydroxyphenyl)-(2Z)-propenoic acid amide (cis-caffeoyltyramine) and/or
n. N-[2-(3,4-dimethoxyphenyl)ethyl]-3-(3,4-dimethoxyphenyl)-(2E)-propenoic acid amide (trans-rubenamine) and/or
o. N-[2-(3,4-dimethoxyphenyl)ethyl]-3-(3,4-dimethoxyphenyl)-(2Z)-propenoic acid amide (cis-rubenamine).

In addition to the aforementioned sharp substances or instead of them, other sharp-tasting substances or those that produce a feeling of heat may also be incorporated into the products according to the invention.

Within the scope of the present invention, alkyl-substituted dioxanes of the following formula have proven to be especially suitable:

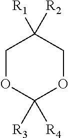

where R₁ and R₂, independently of one another, are selected from —H, —CH₃, —CH₂CH₃ and R₃ and R₄ independently of one another are selected from —H, —CH₃, —CH₂CH₃, —CH₂CH₂CH₃,

### —CH(CH₃)₂.

Within the scope of the present invention, phenyl esters of the following formula have also proven to be especially suitable:

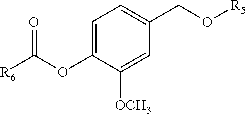

where R₅ stands for —CH₃ or a linear or branched alkyl or alkenyl radical with 2 to 8 carbon atoms and R₆ stands for —CH₃ or a linear or branched alkyl or alkenyl radical with 2 to 8 carbon atoms or an alkoxy group with 1 to 3 carbon atoms.

Within the scope of the present invention, carvone acetals of the following formula have also proven to be especially suitable:

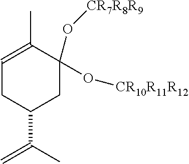

where R₇ to R₁₂, independently of one another, are selected from —H, —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃, or R₉ and R₁₀ together form a chemical bond or denote a group —(CR₁₃R₁₄)ₓ, where x stands for the values 1 or 2, and R₁₃ and R₁₄, independently of one another, are selected from
—H, —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃.

Agents according to the invention can be formulated as toothpastes or dentifrices. Another subject matter of the present invention is the use of agents according to the invention for cleaning teeth by means of electric toothbrushes.

Another subject matter of the present invention is a method for cleaning teeth, characterized in that an agent according to the invention is applied to the brush head of an electric toothbrush and the teeth are cleaned with the electric toothbrush.

Another subject matter is a method for cleaning teeth, which is characterized by the steps:
a) providing a toothbrush whose brush head can be set in motion electrically;
b) applying 0.5 to 5 g of an agent according to the invention to the brush head,
c) brushing the teeth for 30 to 300 seconds using the agent according to the invention with the use of the brush head with its electrically induced motion.

With respect to preferred embodiments of the use and the method according to the invention, what was said about the agents according to the invention is also true mutatis mutandis.

Due to the use of the polylactic acid particles, the effect of the silicates can be increased with respect to cleaning power and can be improved with respect to protection of the surface. Another subject matter of the present invention is therefore the use of polylactic acid particles to increase the cleaning performance and to improve the protection of tooth surfaces by silicate polishing agents.

With respect to preferred embodiments of the use according to the invention, what was said regarding the agents according to the invention also applies mutatis mutandis.

Examples (all amounts given in % by weight):

| | A | B | C | D | E | F |
|---|---|---|---|---|---|---|
| Glycerol | 30.1 | 30.1 | 30.1 | 30.1 | 30.1 | 30.1 |
| Sorbosil AC 35 | 0 | 0 | 0 | 12 | 12 | 12 |
| Sident 9 | 12 | 12 | 12 | 0 | 0 | 0 |
| Sident 22 S | 9 | 9 | 9 | 9 | 9 | 9 |
| Polylactic acid particles (1) | 0.5 | 1.0 | 2.0 | 5.0 | 2.5 | 1.0 |
| Calcium glycerophosphate | 0.13 | 0.2 | 0.5 | 0.13 | 0.2 | 0.5 |
| Disodium phosphate | 0.2 | 0.2 | 0.2 | 0.2 | 0.2 | 0.2 |
| anhydrous | | | | | | |
| Trisodium phosphate | 0.3 | 0.3 | 0.3 | 0.3 | 0.3 | 0.3 |
| anhydrous | | | | | | |
| Sodium saccharin | 0.2 | 0.2 | 0.2 | 0.2 | 0.2 | 0.2 |
| Titanium dioxide | 1 | 1 | 1 | 1 | 1 | 1 |
| Sodium monofluorophosphate | 1.1 | | 1.1 | 1.1 | | 1.1 |
| NaF | | 0.3 | | | 0.3 | |
| Alumina | 0.5 | 0.5 | 0.5 | 0.5 | 0.5 | 0.5 |
| Xanthan FN | 0.7 | 0.7 | 0.7 | 0.7 | 0.7 | 0.7 |
| Sodium lauryl sulfate | 1 | 1 | 1.2 | 1 | 1 | 1.2 |
| Flavoring | 0.95 | 0.95 | 1 | 0.95 | 0.95 | 1 |
| Water | to 100 | to 100 | to 100 | to 100 | to 100 | to 100 |
| | G | H | I | J | K | L |
| Glycerol | 30.1 | 30.1 | 30.1 | 30.1 | 30.1 | 30.1 |
| Sident 9 | 12 | 12 | 12 | 10 | 11 | 10 |
| Sident 22 S | 9 | 9 | 9 | 9 | 9 | 9 |
| Polylactic acid particles (1) | 5.0 | 7.5 | 10.0 | 5.0 | 2.0 | 5.0 |
| Calcium glycerophosphate | 0.13 | 0.2 | 0.5 | 0.13 | 0.2 | 0.5 |
| Sodium saccharin | 0.2 | 0.2 | 0.2 | 0.2 | 0.2 | 0.2 |
| Titanium dioxide | 1 | 1 | 1 | 1 | 1 | 1 |
| Sodium monofluorophosphate | 1.1 | | 1.1 | 1.1 | | 1.1 |
| NaF | | 0.3 | | | 0.3 | |
| Xanthan FN | 0.7 | 0.7 | 0.7 | 0.7 | 0.7 | 0.7 |
| Sodium lauryl sulfate | 1 | 1 | 1.2 | 1 | 1 | 1.2 |
| Flavoring | 0.95 | 0.95 | 1 | 0.95 | 0.95 | 1 |
| Water | to 100 | to 100 | to 100 | to 100 | to 100 | to 100 |

(1): Ecoscrub ® 50 PC, maximum particle size 297 μm

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An oral and dental hygiene and cleaning agent comprising, based on its weight,
a) 0.001 to 25% by weight polylactic acid particles, and
b) 1 to 30% by weight precipitated silicic acid(s) with a specific surface area according to ISO 5794-1, Appendix D of ≦60 m²/g.

2. The oral and dental hygiene and cleaning agent according to claim 1, wherein the polylactic acid particles comprise, 0.002-20% by weight based on the total weight of the agent.

3. The oral and dental hygiene and cleaning agent according to claim 1, wherein the polylactic acid particles comprise 0.005-12.5% by weight based on the total weight of the agent.

4. The oral and dental hygiene and cleaning agent according to claim 1, wherein the polylactic acid particles have particle sizes of 1 to 1000 μm.

5. The oral and dental hygiene and cleaning agents according to claim 1, wherein the polylactic acid particles have particle sizes of 2 to 750 μm.

6. The oral and dental hygiene and cleaning agent according to claim 1, wherein the polylactic acid particles have particle sizes of 10 to 500 μm.

7. The oral and dental hygiene and cleaning agent according to claim 1, wherein the polylactic acid particles have average ECD values of 10 to 1000 μm.

8. The oral and dental hygiene and cleaning agent according to claim 1, wherein the polylactic acid particles have average ECD values 100 to 350 μm.

9. The oral and dental hygiene and cleaning agents according to claim 1, wherein the polylactic acid particles have an average solidity of 0.4 to 0.9.

10. The oral and dental hygiene and cleaning agent according to claim 1, wherein the polylactic acid particles have an average solidity of 0.55 to 0.65.

11. The oral and dental hygiene and cleaning agent according to claim 1, wherein the polylactic acid particles have an average circularity C of 0.1 to 0.6.

12. The oral and dental hygiene and cleaning agent according to claim 1, wherein the polylactic acid particles have an average circularity C of 0.2 to 0.35.

13. The oral and dental hygiene and cleaning agent according to any claim 1, wherein the precipitated silicic acids with a specific surface area according to ISO 5794-1, Appendix D of <55 m²/g comprises 2.5 to 25% by weight based on the total weight of the agent.

14. The oral and dental hygiene and cleaning agent according to any claim 1, wherein the precipitated silicic acids with a specific surface area according to ISO 5794-1, Appendix D of <55 m²/g comprises 8 to 15% by weight based on the total weight of the agent.

15. The oral and dental hygiene and cleaning agent according to claim 1, wherein all the precipitated silicic acid(s) present has a specific surface area according to ISO 5794-1, Appendix D of ≦60 m²/g.

16. The oral and dental hygiene and cleaning agent according to claim 1, further comprising 0.01 to 2.5% by weight calcium glycerophosphate.

17. The oral and dental hygiene and cleaning agent according to claim 1, further comprising 0.11 to 0.75% by weight calcium glycerophosphate.

18. The oral and dental hygiene and cleaning agent according to claim 1, further comprising 0.1 to 7.5% by weight xanthan.

19. The oral and dental hygiene and cleaning agent according to claim 1, wherein the agent comprises, based on its weight, less than 5% by weight surfactant.

20. Method for cleaning teeth, characterized by the steps:
a) providing a toothbrush whose brush head can be induced electrically to movement;
b) applying 0.5 to 5 g of an agent according to claim 1 to the brush head,
c) tooth brushing for 30 to 300 seconds using the agent.
